(19) United States
(12) Patent Application Publication
(10) Pub. No.: US 2013/0306078 A1
(43) Pub. Date: Nov. 21, 2013

(54) Nasal Cannula Cover

(71) Applicant: Tracy Lanciotto, Rocky Hill, CT (US)

(72) Inventor: Tracy Lanciotto, Rocky Hill, CT (US)

(21) Appl. No.: 13/898,515

(22) Filed: May 21, 2013

Related U.S. Application Data
(60) Provisional application No. 61/649,475, filed on May 21, 2012.

(51) Int. Cl. A61M 16/06

(52) U.S. Cl. CPC A61M 16/0666
USPC 128/207.18

(57) ABSTRACT

A nasal cannula cover is provided and includes a cover body having a cover front, a cover rear, a cover length and cover width, wherein the cover length and cover width are sized to cover at least a portion of a nasal cannula having a cannula oxygen output port, wherein the cover body further includes, at least one oxygen output port opening for receiving the cannula oxygen output port, and a body connection device having a first attaching portion and a second attaching portion, wherein when the cover body is associated with the nasal cannula,the first attaching portion and second attaching portion are configured to securely and removably engage with each other, and the cannula oxygen output port of the nasal cannula is protruding from the at least one oxygen output port opening

### RELATED APPLICATIONS

This application claims priority to and benefit of the filing date of U.S. Provisional Patent Application Ser. No. 61/649,475 (Atty. Docket No. LAN-0001-P), filed May 21, 2012, the contents of which are incorporated by reference herein in its entirety.

### FIELD OF THE INVENTION

This invention relates generally to a nasal tube and nasal cannula and more particularly to cover for a nasal tube and nasal cannula.

### BACKGROUND OF THE INVENTION

Nasal cannulas are well known and are used to deliver oxygen to the nasal cavity of an oxygen compromised person, where the person may be mobile or the person may be confined to a bed in a hospital or other health care setting. For example, elderly people with Chronic Obstructive Pulmonary Disease (COPD), such as bronchitis, or emphysema, may carry around an oxygen bottle which is connected to a tube that delivers oxygen to the nasal cavity via a tube/cannula combination. As another example, a patient who is confined to a hospital bed may also require the delivery of oxygen. Referring to FIG. 1, FIG. 2 and FIG. 3, one such cannula setup is shown and typically includes a nasal cannula 100 having a hollow cannula tube 102 with two hollow protrusions 104, wherein the hollow cannula tube 102 typically includes a cannula opening 103 on each end of the cannula tube 102. Each of the hollow protrusions 104 include an oxygen output opening 106 located proximate the end of the protrusion 104, wherein the hollow tube 102 is connected to an oxygen source 108 via an oxygen tube 110 which is in flow communication with the hollow cannula tube 102 via the cannula openings 103 on each end of the cannula tube 102. When being used, oxygen flows from the oxygen source 108, through the oxygen tube 110 and into hollow cannula tube 102 of the cannula 100, where the oxygen flows out of the oxygen output openings 106 and into the nose of a wearer. It should be appreciated that the hollow protrusions 104 typically extend away from the hollow tube 102 far enough to be located at least partially within (or at least proximate) the nose/nostril of the wearer. The airflow is thus directed into the nostril of the wearer via the oxygen output openings 106 located on or near the ends of the hollow protrusion 104.

Unfortunately, one substantial problem that arises with people who use nasal cannulas for extended (and even short) periods of time is that the skin beneath the hollow cannula tube 102 and/or the oxygen tube 110 may become irritated due to the rubbing of the hollow cannula tube 102 and/or the oxygen tube 110 on the skin. As a result, the skin beneath the nose and along the upper lip and cheek area of the wearer (i.e. in the areas that come in contact with the hollow cannula tube 102 and/or the oxygen tube 110 are subject to irritation and sores. This is undesirable for at least two reasons. First, people who are oxygen compromised are reticent to wear the cannula as often as they should due to the discomfort caused by the rubbing. Second, people who are oxygen compromised also tend to have a less robust immune system, which makes them more subject to sores, slower healing and opportunistic infections.

### SUMMARY OF THE INVENTION

A nasal cannula cover is provided and includes a cover body having a cover front, a cover rear, a cover length and cover width, wherein the cover length and cover width are sized to cover at least a portion of a nasal cannula having a cannula oxygen output port, wherein the cover body further includes, at least one oxygen output port opening for receiving the cannula oxygen output port, and a body connection device having a first attaching portion and a second attaching portion, wherein when the cover body is associated with the nasal cannula,the first attaching portion and second attaching portion are configured to securely and removably engage with each other, and the cannula oxygen output port of the nasal cannula is protruding from the at least one oxygen output port opening.

A nasal cannula cover is provided and includes a cover body having a cover front, a cover rear, a cover length and cover width, wherein the cover length and cover width are sized to cover at least a portion of a nasal cannula having a cannula oxygen output port, wherein the cover body further includes, at least one oxygen output port opening for receiving the cannula oxygen output port, and wherein when the cover body is associated with the nasal cannula, the cover body is wrapped around the nasal cannula and the cannula oxygen output port of the nasal cannula is protruding from the at least one oxygen output port opening.

### BRIEF DESCRIPTION OF THE DRAWINGS

The foregoing and other features and advantages of the present invention will be more fully understood from the following detailed description of illustrative embodiments, taken in conjunction with the accompanying drawings in which like elements are numbered alike:

FIG. 1 is a front view of a cannula attached to an oxygen tank via an oxygen tube, in accordance with the prior art.

FIG. 2 is a top perspective view of a cannula, in accordance with the prior art.

FIG. 3 is front view of a person wearing a cannula that is attached to an oxygen tank via an oxygen tube, in accordance with the prior art.

FIG. 4a is a front view of a cover body for a nasal cannula cover, in accordance with one embodiment of the present invention.

FIG. 4b is a rear view of a cover body for the nasal cannula cover of FIG. 4a.

FIG. 4c is a side view of a cover body for the nasal cannula cover of FIG. 4a.

FIG. 5a is a side view of the cover body of the nasal cannula cover of FIG. 4a, in accordance with one embodiment of the present invention.

FIG. 5b is a side view of a hollow tube of the nasal cannula of FIG. 1.

FIG. 5c is a side view of the cover body of FIG. 5a associated with the hollow tube of FIG. 1, in accordance with one embodiment of the present invention.

FIG. 6 is a front view of the nasal cannula cover of FIG. 4a assocated with the cannula of FIG. 1, in accordance with one embodiment of the present invention.

FIG. 7a is a front view of a cover body for a nasal cannula cover, in accordance with another embodiment of the present invention.

FIG. 7b is a rear view of a cover body for the nasal cannula cover of FIG. 7a.

FIG. 7c is a side view of a cover body for the nasal cannula cover of FIG. 7a.

FIG. 8a is a front view of a cover body for a nasal cannula cover, in accordance with still yet another embodiment of the present invention.

FIG. 8b is a rear view of a cover body for the nasal cannula cover of FIG. 8a.

FIG. 8c is a side view of a cover body for the nasal cannula cover of FIG. 8a.

FIG. 9a is a front view of a cover body for a nasal cannula cover, in accordance with still yet another embodiment of the present invention.

FIG. 9b is a rear view of a cover body for the nasal cannula cover of FIG. 9a.

FIG. 9c is a side view of a cover body for the nasal cannula cover of FIG. 9a.

FIG. 10 is a side view of the cover body of FIG. 9a associated with the nasal cannula of FIG. 1, in accordance with another embodiment of the present invention.

FIG. 11 is a bottom up view of the cover body of FIG. 9a associated with the nasal cannula of FIG. 1 being worn by a user, in accordance with another embodiment of the present invention.

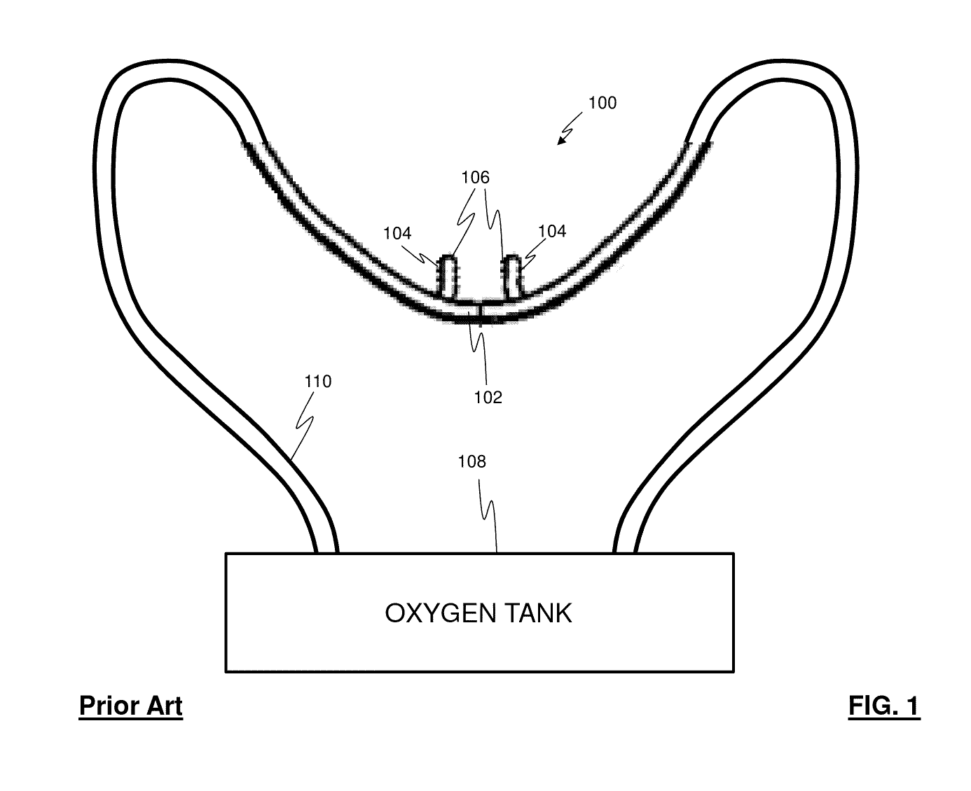

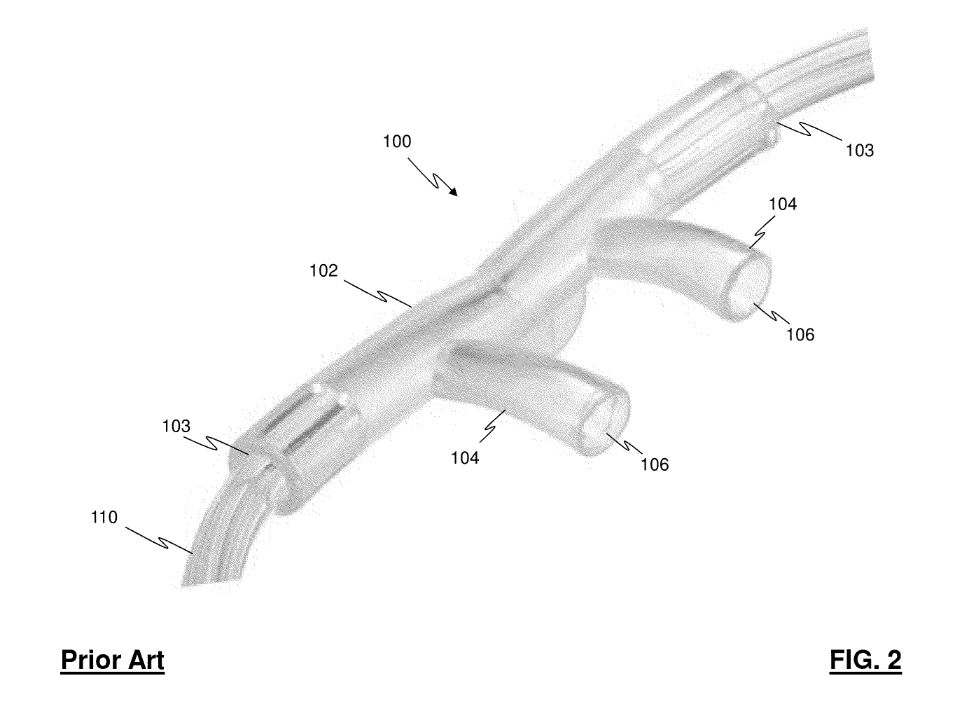

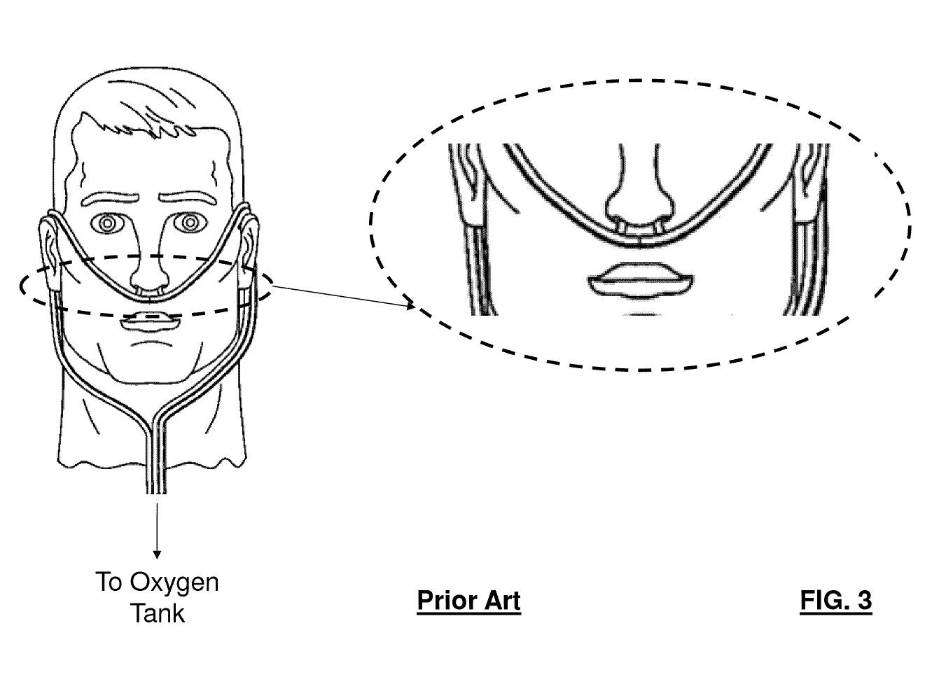

### DETAILED DESCRIPTION OF THE INVENTION

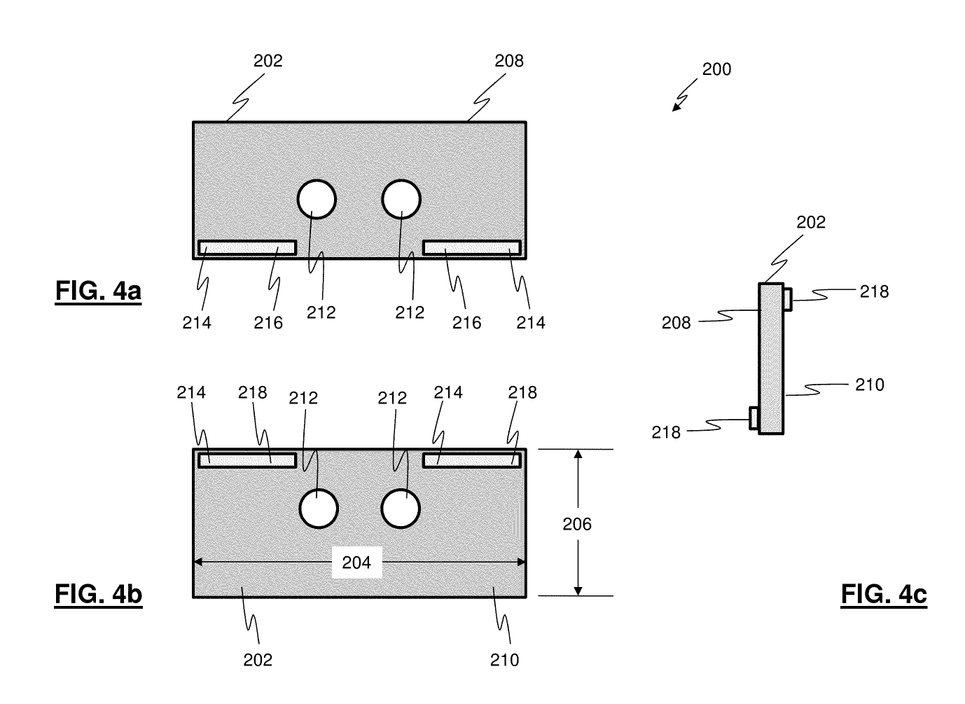

In accordance with one embodiment of the present invention, referring to FIG. 4a, FIG. 4b and FIG. 4c a nasal cannula cover 200 is provided and includes a cover body structure 202 having a cover length 204 and a cover width 206. The cover body 202 further includes a cover front 208, a cover rear 210 and at least one oxygen output port opening 212 (shown as two spaced apart openings). The cover body 202 also includes an attaching member 214 for attaching the cover body 202 to the hollow tube 102 of a nasal cannula 100, wherein the attaching member 214 includes a first attaching portion 216 located on the cover front 208 and a second attaching portion 218 located on the cover rear 210. In accordance with the present invention, the attaching member 214 may be any attaching member suitable to the desired end purpose, such as Velcro®, snaps and/or zipper. The cover body 202 is preferably constructed from a soft, hypoallergenic material that is washable (or disposable/replaceable).

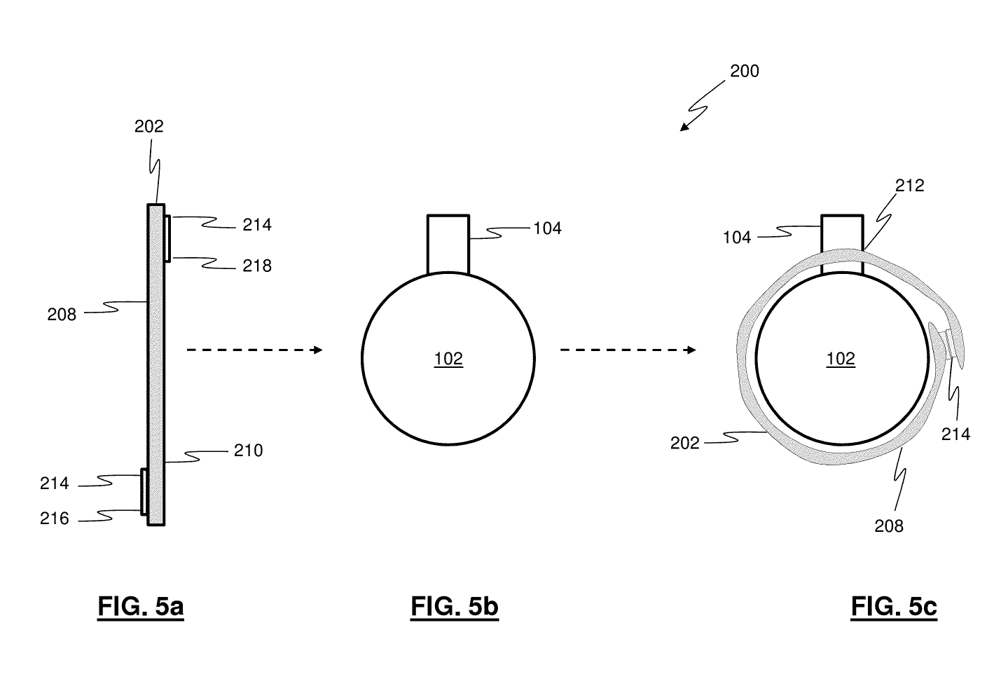

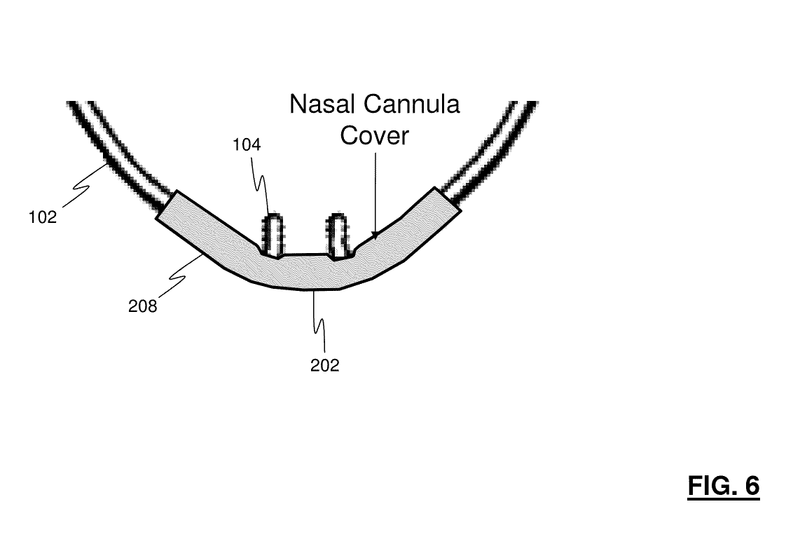

As shown in FIG. 5a, FIG. 5b and FIG. 5c, the nasal cannula cover 200 is attached to the hollow tube 102 of a nasal cannula 100 by wrapping the cover body 202 around the hollow tube 102 such that the cover rear 210 is proximate the hollow tube 102 and such that the hollow protrusions 104 having the oxygen output openings 106 protrude through the at least one oxygen output port opening 212 on the cover body 202. The first attaching portion 216 is then associated with the second attaching portion 218 such that the attaching portions 216, 218 securingly interact with each other to securely cover the nasal cannula cover 200, as shown in FIG. 5c and FIG. 6. Accordingly, when worn by a person, the cover front 208 is proximate the facial skin of the wearer.

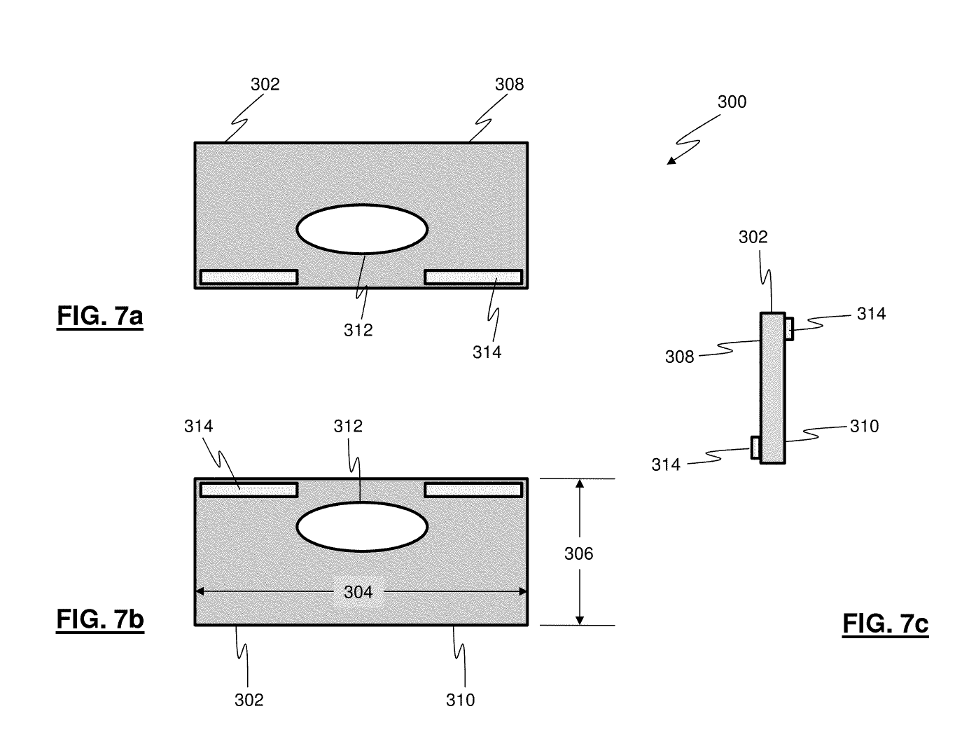

In accordance with another embodiment of the present invention, referring to FIG. 7a, FIG. 7b and FIG. 7c, a nasal cannula cover 300 is provided and includes a cover body 302 having a cover length 304 and a cover width 306. The cover body 302 further includes a cover front 308, a cover rear 310 and at least one oxygen output port opening 312. The cover body 302 also includes an attaching member 314 for attaching the cover body 302 to the hollow tube 102 of a nasal cannula 100, wherein the attaching member 314 includes a first attaching portion 316 located on the cover front 308 and a second attaching portion 318 located on the cover rear 310 similar to that discussed hereinabove. The attaching member 314 may be any attaching member suitable to the desired end purpose, such as Velcro®, snaps and/or zipper. The cover body 302 is preferably constructed from a soft, hypoallergenic material that is washable (or disposable/replaceable).

The cover body 302 is associated with the hollow tube 102 of a nasal cannula 100 in a similar fashion as described hereinabove and as shown in FIG. 5. For example, the nasal cannula cover 300 is attached to the hollow tube 102 of a nasal cannula 100 by wrapping the cover body 302 around the hollow tube 102 such that the cover rear 310 is proximate the hollow tube 102 and such that the hollow protrusions 104 having the oxygen output openings 106 protrude through the oxygen output port opening 312 on the cover body 302. The first attaching portion 316 is associated with the second attaching portion 318 such that the attaching portions 316, 318 securingly interact with each other.

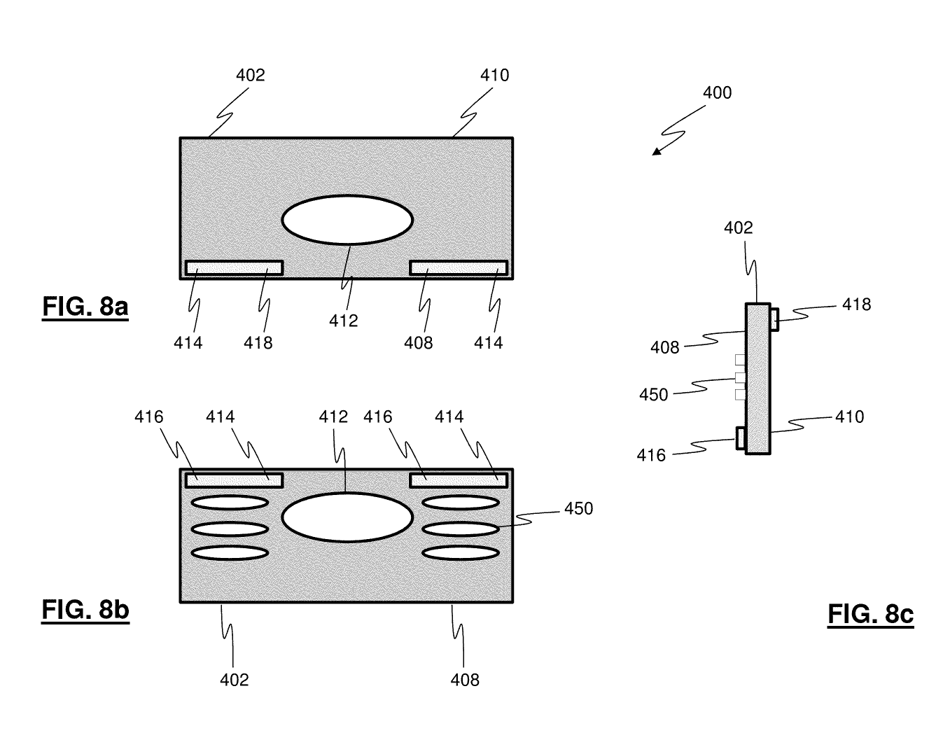

Referring to FIG. 8a, FIG. 8b and FIG. 8c, in accordance with still yet another embodiment, a nasal cannula cover 400 is provided and includes a cover body 402 having a cover length 404 and a cover width 406. The cover body 402 further includes a cover front 408, a cover rear 410 and at least one oxygen output port opening 412. The cover body 402 also includes an attaching member 414 for attaching the cover body 402 to the hollow tube 102 of a nasal cannula 100, wherein the attaching member 414 includes a first attaching portion 416 located on the cover front 408 and a second attaching portion 418 located on the cover rear 410. The cover body 402 may include one or more channels/pockets 450 communicated with the cover front 408, where the channels 450 may be configured to receive and contain a lotion or other skin treatment medicine to help prevent skin rash or sores from occurring. Additionally, it is contemplated that the channels/pockets 450 may be configured to receive and contain a substance, such as a menthol vapor product, for helping the wearer breath better. In still yet another embodiment, a nasal cannula cover 500 is provided and includes a cover body 502 that is hollow for receiving a pillow type of device for increasing the comfort of the wearer.

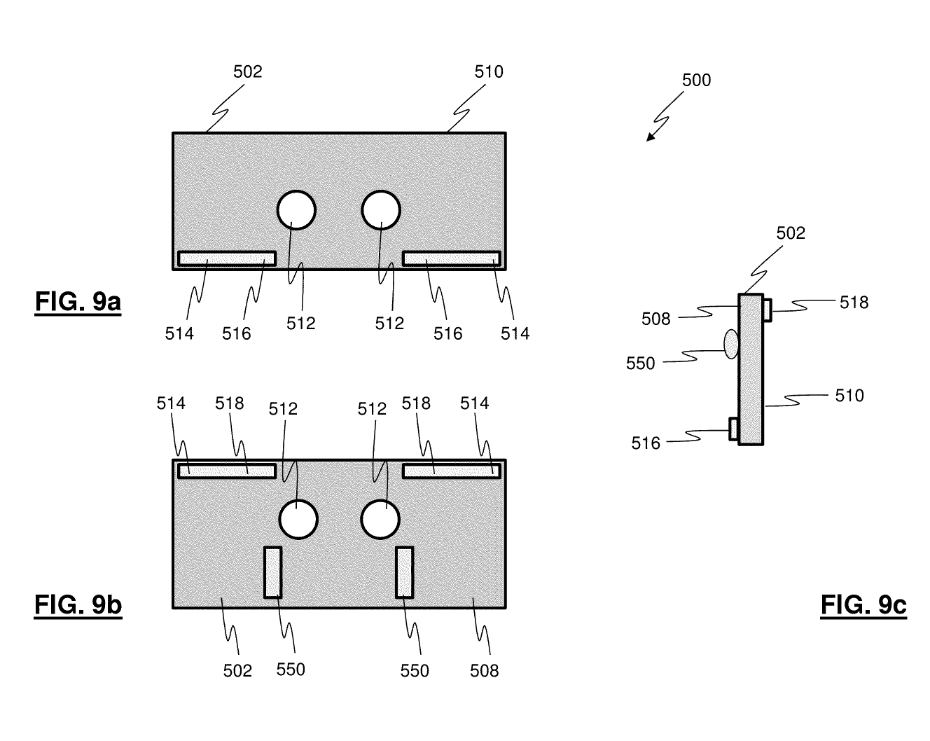

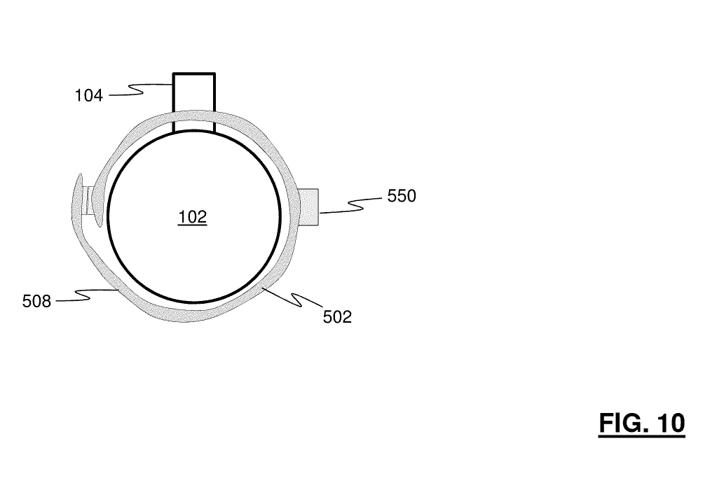

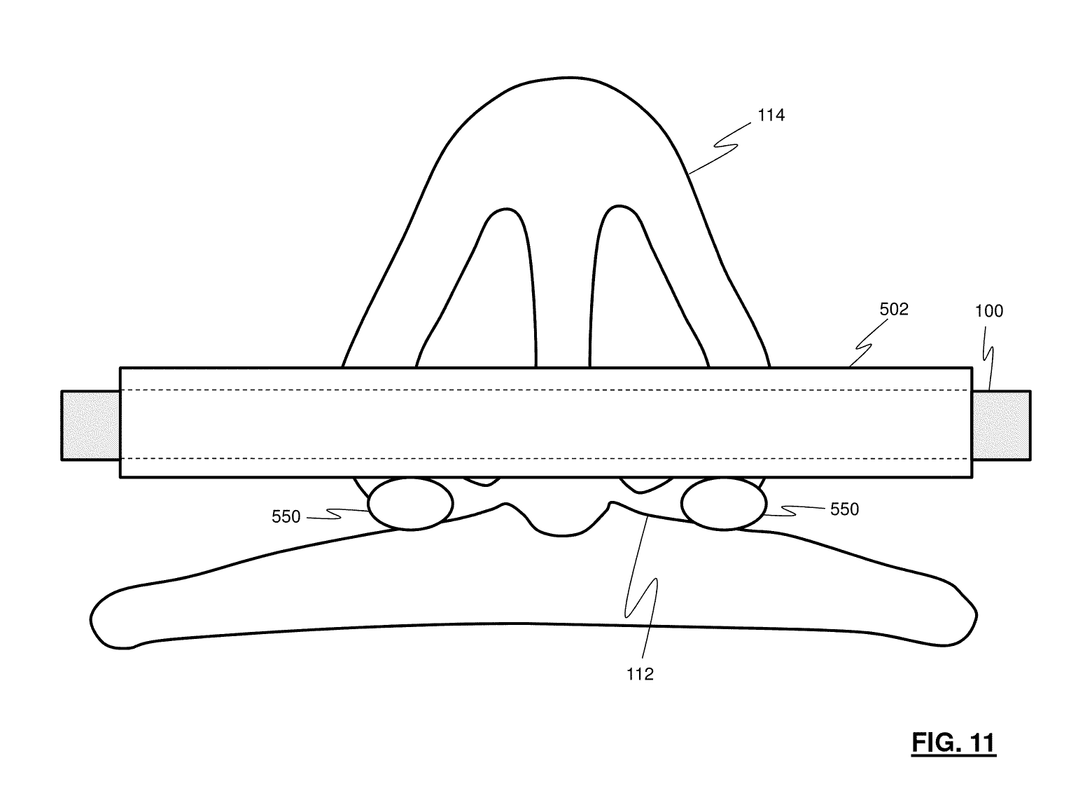

Referring to FIG. 9a, FIG. 9b and FIG. 9c, in accordance with still yet another embodiment, a nasal cannula cover 500 is provided and includes a cover body 502 having a cover length 504 and a cover width 506. The cover body 502 further includes a cover front 508, a cover rear 510 and at least one oxygen output port opening 512. The cover body 502 also includes an attaching member 514 for attaching the cover body 502 to the hollow tube 102 of a nasal cannula 100, wherein the attaching member 514 includes a first attaching portion 516 located on the cover front 508 and a second attaching portion 518 located on the cover rear 510. The cover body 502 may include one or more body ridges 550 located on the cover front 508, where the body ridge 550 may be located on the front cover 508 to be between the cannula 100 and the face of the wearer, thus keeping the cover away from the skin of the wearer's face on the majority of the surface of the wearer. This is shown in FIG. 10. In this way, the contact between the cover 502 and the wearers face will be minimized to help prevent skin rash or sores from occurring. Referring to FIG. 11, the nasal cannula cover 500 is shown on a nasal cannula being worn by a user. As can be seen the body ridges 550 of the cover body 502 keep most of the nasal cannula 100 away from the skin of the wearer (i.e the skin between the upper lip 112 and the nose 114 of the wearer) to minimize contact and thus potential for irritation.

It is contemplated that in yet another embodiment, the body structure 202, 302, 402, 502 may be configured without an attaching member and may include an inner structure (rigid or semi-rigid) that is resilient and naturally ‘folds’ in on itself and around the hollow tube 102 of a nasal cannula 100. Thus, the inner structure may have a ‘memory’ such that when in its natural configuration the sides fold in on themselves to form a cylindrical shaped body. Thus, the sides of the cover body 202, 302, 402, 502 are spread apart to wrap around hollow tube 102 of the nasal cannula 100. And because the natural configuration of the body structure 202, 302, 402, 502 is cylindrical shaped, there is no need for an attaching portion.

In still yet another embodiment, the cover body 202, 302, 402, 502 may be configured without an attaching member 214, 314, 314, 514 and may be configured as a sleeve that slides over the nasal cannula 100 (such as a sock like configuration), where the cover body 202, 302, 402, 502 may or may not be constructed from a resilient or elastic material.

While the invention has been described with reference to an exemplary embodiment, it will be understood by those skilled in the art that various changes, omissions and/or additions may be made and equivalents may be substituted for elements thereof without departing from the spirit and scope of the invention. In addition, many modifications may be made to adapt a particular situation or material to the teachings of the invention without departing from the scope thereof. Therefore, it is intended that the invention not be limited to the particular embodiment disclosed as the best mode contemplated for carrying out this invention, but that the invention will include all embodiments falling within the scope of the appended claims. Moreover, unless specifically stated any use of the terms first, second, etc. do not denote any order or importance, but rather the terms first, second, etc. are used to distinguish one element from another.

What is claimed is:

1. A nasal cannula cover, comprising,
a cover body having a cover front, a cover rear, a cover length and cover width, wherein the cover length and cover width are sized to cover at least a portion of a nasal cannula having a cannula oxygen output port, wherein the cover body further includes,
at least one oxygen output port opening for receiving the cannula oxygen output port, and
a body connection device having a first attaching portion and a second attaching portion, wherein when the cover body is associated with the nasal cannula,
the first attaching portion and second attaching portion are configured to securely and removably engage with each other, and the cannula oxygen output port of the nasal cannula is protruding from the at least one oxygen output port opening.

2. The nasal cannula cover of claim 1, wherein the nasal cannula cover is constructed from a soft, washable, hypoallergenic material.

3. The nasal cannula cover of claim 1, wherein the first attaching device and second attaching device is at least one of Velcro®, a snap and a zipper.

4. The nasal cannula cover of claim 1, wherein the at least one oxygen output port opening includes two oxygen output port openings.

5. The nasal cannula cover of claim 1, wherein when the nasal cannula cover is associated with the nasal cannula, the cannula oxygen output port of the nasal cannula is protruding from the at least one oxygen output port opening.

6. The nasal cannula cover of claim 1, further comprising a channel/pocket, wherein the channel/pocket is located on the cover front and is configured to contain a lotion.

7. The nasal cannula cover of claim 1, further comprising a body ridge, wherein the body ridge is located on the cover front and is configured to located the cover front away from a wearer's face.

8. A nasal cannula cover, comprising,
a cover body having a cover front, a cover rear, a cover length and cover width, wherein the cover length and cover width are sized to cover at least a portion of a nasal cannula having a cannula oxygen output port, wherein the cover body further includes,
at least one oxygen output port opening for receiving the cannula oxygen output port, and
wherein when the cover body is associated with the nasal cannula, the cover body is wrapped around the nasal cannula and the cannula oxygen output port of the nasal cannula is protruding from the at least one oxygen output port opening.

9. The nasal cannula cover of claim 8, further comprising a first attaching portion and a second attaching portion, wherein the first attaching portion and second attaching portion are configured to securely and removably associate with each other.

10. The nasal cannula cover of claim 8, further comprising a resilient body structure such that the body structure is configurable between a cylindrical shape and a non-cylindrical shape, wherein when associated with the nasal cannula and configured in the cylindrical shape, the body structure is securely wrapped around the nasal cannula.

11. The nasal cannula cover of claim 8, wherein the nasal cannula cover is constructed from a soft, washable, hypoallergenic material.

12. The nasal cannula cover of claim 8, wherein the first attaching device and second attaching device is at least one of Velcro®, a snap and a zipper.

13. The nasal cannula cover of claim 8, wherein the at least one oxygen output port opening includes two oxygen output port openings.

14. The nasal cannula cover of claim 8, further comprising a channel/pocket, wherein the channel/pocket is located on the cover front and is configured to contain a lotion.

15. The nasal cannula cover of claim 8, further comprising a body ridge, wherein the body ridge is located on the cover front and is configured to located the cover front away from a wearer's face.
